(19) United States
(12) Patent Application Publication
(10) Pub. No.: US 2002/0172955 A1
(43) Pub. Date: Nov. 21, 2002

(54) Methods for purifying DNA using immobilized capture probes

(75) Inventors: Christopher P. Adams, Somerville, MA (US); T. Christian Boles, Waltham, MA (US); Lawrence Weir, Hopkinton, MA (US); Rahul K. Dhanda, Somerville, MA (US); Nevin M. Summers, Brookline, MA (US)

Correspondence Address: TESTA, HURWITZ & THIBEAULT, LLP, HIGH STREET TOWER, 125 HIGH STREET, BOSTON, MA, 02110, US

(73) Assignee: EXACT Sciences Corporation, Maynard, MA (US)

(21) Appl. No.: 09/939,275

(22) Filed: Aug. 24, 2001

Related U.S. Application Data
(63) Continuation of application No. 09/259,467, filed on Feb. 26, 1999, now abandoned.
Application No. 09/259,467 is a continuation-in-part of application No. PCT/US98/09952, filed on May 15, 1998.
Application No. 09/259,467 is a continuation-in-part of application No. 08/971,845, filed on Aug. 8, 1997.
(60) Provisional application No. 60/046,708, filed on May 16, 1997.

(51) Int. Cl. C12Q 1/68

(52) U.S. Cl. 435/6; 204/464

(57) ABSTRACT

Methods employing purification devices comprising an electrophoretic medium containing immobilized capture probes for the purification of DNA are disclosed.

### RELATED APPLICATION

This application is a continuation of U.S. application Ser. No. 09/259,467 filed Feb. 26, 1999 which is a continuation-in-part of and claims priority to PCT/US98/09952, filed May 15, 1998, and U.S. application Ser. No. 08/971,845, filed Aug. 8, 1997, which claims the benefit of U.S. Provisional Application No. 60/046,708, filed May 16, 1997. The teachings of all the above referenced applications are hereby incorporated by reference in their entireties.

### BACKGROUND OF THE INVENTION

Nucleic acid sequence information plays a vital role in both basic and applied biomedical research. The nucleotide sequence of a particular portion of DNA can be instructive as to the molecular basis for a given disease, such as Huntington's Disease. Once a segment of genome has been identified as being potentially responsible for a particular affliction, elucidating the nucleotide sequence becomes very important. The sequence, once known, can play a part in the therapeutic regime to be provided, such as in the case of gene therapy. This is most evident when the basis of the disease is a genetic mutation of the normal gene. One methodology employed for treating genetic mutation-based diseases is the introduction of the wild-type nucleotide sequence. But first it must be established that in fact a gene, or an aberrant form of a gene, is the etiologic agent for a particular disease or syndrome. This information is most often provided through the isolation and characterization of the putative aberrant gene. Characterization often involves the sequence analysis of the nucleotide sequence itself that defines the gene of interest. This will often involve understanding both the wild-type, or physiologically normal, and mutant genes.

In practice, the quality of the sequence analysis is, in part, a reflection of the quality of the starting material. It is vital that the preparation that is to be subjected to sequence analysis be of high quality, that is, relatively pure and free of contaminating species like proteins and small molecules, such as salts, that can interfere with obtaining a high quality result from sequence analysis. Current protocols involve ethanol precipitation in order to remove unincorporated nucleotides and salt from, for example, extension products prior to sequence analysis. Also, with regard to the extension products, it is often desirable to remove any template DNA and excess primers from the preparation prior to sequence analysis. Precipitation is time consuming and requires care to achieve consistent product yields. Nevertheless, it is critical to the success of performing an informative sequence analysis on a target nucleotide sequence that the target sequence preparation be as free of contaminating molecules as possible. Additionally, it would be advantageous to have a purification system that could sort the products of multiplexed sequencing reactions.

### SUMMARY OF THE INVENTION

The present invention pertains to methods of purifying a target molecule contained within a test sample. Typically, the target molecule in a test sample will be a nucleic acid molecule, in particular, single-stranded DNA primer extension sequence products of a dideoxy sequencing reaction, for example, the Sanger method, Sanger, F., et al., Proc. Natl. Acad. Sci. USA, 74:5463-5467 (1977), or a cycle sequencing method, Carothers, Biotechniques,, 7:494-499 (1989), the entire teachings of which are herein incorporated by reference in their entirety. Once purified, these purified nucleic acid molecules can be used in a variety of ways including being subjected to capillary or slab gel electrophoresis for DNA sequence analysis. Nucleic acid molecule capture probes modified with 5′-acrylamide groups are copolymerized within an electrophoresis gel, such as a polyacrylamide gel. Single-stranded target nucleic acid molecules can bind to their complementary sequence contained within a capture probe, if there is sufficient complementarity between the two molecules. Double-stranded target nucleic acid molecules can bind to a complementary sequence forming a triple helical arrangement.

The target molecule, or molecules, present in a test sample can be placed in an electrophoresis gel containing immobilized capture probes and undergo electrophoresis. The target molecule will migrate through the gel medium until it comes in contact with its complementary immobilized capture probe. Once the target and capture probe are in contact with one another, they can hybridize forming a complex. The non-target molecules contained in the test sample can continue electrophoresis and are effectively removed from the target molecule.

In one embodiment of the present invention, the target molecules are DNA extension products formed during a primer extension sequencing reaction. A reaction mixture from a primer extension sequencing reaction is loaded into a purification device, for example, a microtiter plate containing multiple wells (having, e.g., 6, 12, 96 or 384 wells). The purification device comprises an electrophoresis gel containing immobilized capture probes that are complementary to at least one nucleotide sequence region contained within the target molecules. Preferably, an electric field is applied such that all negatively charged molecules migrate through the electrophoresis gel toward the a positively charged electrode. The positively charged electrode can be housed in a positively charged electrode buffer chamber. This chamber can be used to collect molecules that exit the electrophoretic medium as a result of their electrophoretic migration. The target molecules will be captured by complementary, immobilized capture probes that are within the gel. The non-target molecules contained within the test sample will pass through the gel and into the positively charged electrode buffer (also referred to herein as the collecting chamber). The collecting chamber can then be replaced with fresh positively charged electrode buffer. A sufficient voltage can be applied so as to denature the hybridization complex formed between the target molecule and capture probe thereby releasing the target molecule. The electric field can be applied using the same polarity as originally applied, thereby allowing for the continued migration of the released target molecule into the collecting chamber containing fresh positively charged electrode buffer. Alternatively, the electric field can be reversed drawing the released target molecule back into the test sample well of the purification device. The purified target molecule can now be accessed and subjected to further analysis, such as capillary or slab gel electrophoresis for sequence analysis.

### BRIEF DESCRIPTION OF THE DRAWINGS

FIG. 1 is a schematic representation of purifying a target nucleic acid molecule from a extension sequencing reaction using an electrophoresis gel with capture probes immobilized within a region of the gel.

FIG. 2 is a schematic representation of the steps involved in purifying extension products using a microtiter well comprising an electrophoretic medium containing capture probes immobilized within the medium.

FIG. 3 is the organization of sequencing and capture primers relative to the template, M13mp18.

FIG. 4 is a schematic drawing illustrating the experimental design for DNA isolation using an electrophoretic medium.

FIG. 5 is shows the effects of varying the elution voltage.

FIG. 6 is shows results obtained from subjecting extension sequencing products to electrophoresis in which the electrophoretic medium contained immobilized capture probes;

FIG. 6a shows the results of the experiment after running the gel for thirty minutes;

FIG. 6b shows the results of the experiment after sixty minutes.

### DETAILED DESCRIPTION OF THE INVENTION

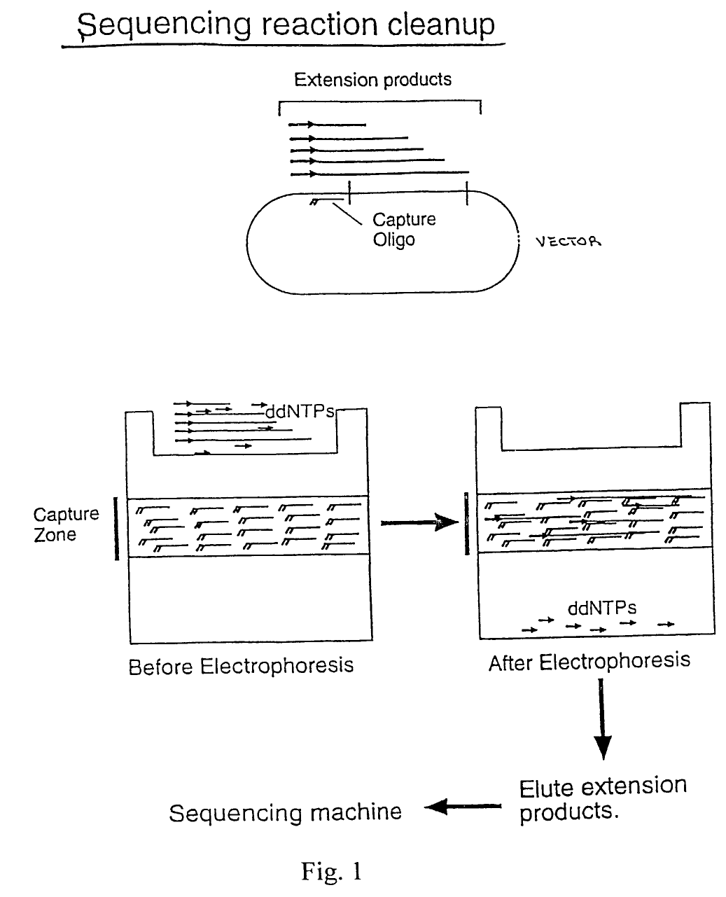

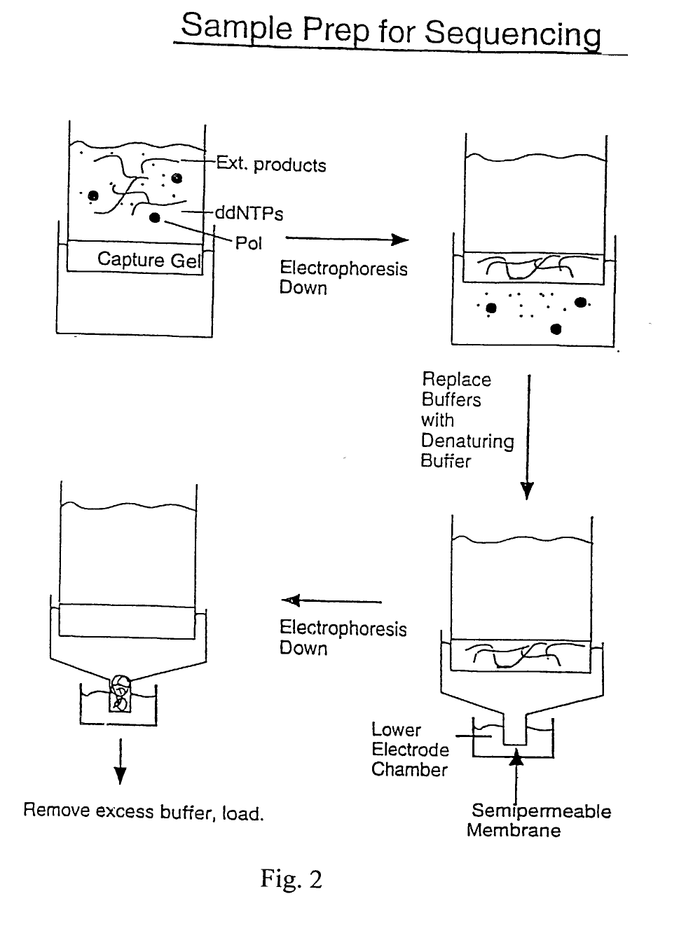

The present invention provides both methods and devices employed to purify a test sample containing a target molecule. It should be understood that the use of the singular term “target molecule” is only used for simplicity throughout this application, and the plural form “target molecules” is implied therein. The methods described herein employ nucleic acid molecule (e.g., oligonucleotide) capture probes immobilized within an electrophoretic medium. The capture probe can be dispersed throughout the electrophoretic medium, or immobilized within discrete layers of the medium as described in U.S. Ser. No. 08/971,845, the entire teaching of which is incorporated herein by reference in its entirety. This electrophoretic medium is contained within a purification device, such as a microtiter plate. The capture probes can be designed to specifically interact with, and hybridize to, a target molecule contained within a test sample. The test sample comprising target and non-target molecules can be introduced into the device, for example, a microtiter plate comprising an electrophoretic medium containing immobilized capture probes. An electric field can be applied to the purification device so that charged molecules in the test sample will migrate within the electrophoretic medium toward the appropriate pole. For example, for the device in the Exemplification, voltages for capture would fall in the range of 0.1 to 200 V, more preferably, between 50 and 150 V. Typically, the molecules of interest will possess a negative charge and therefore migrate toward the positively charged electrode. The target molecule will continue to migrate until it has come into contact with an immobilized capture probe which is specific for that particular target molecule. A hybridization complex can then form between the immobilized capture probe and target molecule. (See FIG. 1). This hybridization complex prevents further migration of the target molecule and allows for the continued migration of non-target molecules, thereby effectuating purification of the target molecule contained within the test sample. Non-target molecules can include proteins, such as enzymes, small molecules like salts, non-targeted nucleotides as well as other non-target molecules, that is, those molecules not targeted for further processing. (See FIG. 2). The target molecule can subsequently be released from the capture probe by applying a sufficient voltage and exit the gel for further analysis. The target molecule can be released from the capture probe by applying a sufficient voltage. For example, using the device in the Exemplification, the target molecule can be eluted from the capture layer (i.e., that layer in the electrophoresis medium containing the immobilized capture probes) at voltages of 250V or higher. For example, for the device in the Exemplification, voltages for elution would be in the range of 250 to 1000 V, more preferably, 250 to 300 V. Suitable voltages for capture and elution using the purification devices described herein can be easily determined by one of skill in the art.

The methods of the present invention use a purification device which comprises three regions. The first region comprises a test sample receptacle which receives a given test sample. The test sample receptacle can be positioned in such a manner as to be proximal to at least one orifice that allows for the delivery of a test sample (e.g., a reaction mixture from a primer extension sequencing reaction). In one embodiment, this orifice is the opening at the top of a microtiter well. The second region of the purification device comprises an electrophoretic medium. Preferably, the electrophoretic medium comprises capture probes immobilized within the medium. Preferably, this second region is physically positioned adjacent to the first region. In one embodiment, the second region is positioned basally to the first position and is also adjacent to the first region. In a preferred embodiment, this second region is formed within one, or more, microtiter wells, though still allowing for the first region to receive and store test sample. The third region of the purification device can be physically contiguous with, or attached to the second region of the purification device. This third region can house a chamber that can collect molecules that exit the second region, in this instance the chamber is referred to as a collecting chamber. The chamber can also perform other functions such as to house buffer. The purification device can also be attached to, or have the capacity to connect with, a power source which generates DC voltage (e.g., a battery).

In one embodiment, the purification device is a microtiter plate containing a set of multiple wells, for example 6, 12, 48, 96 or 384 wells. The well, or wells, of the microtiter plate comprises the three regions elucidated above for the purification device. The first region comprises a test sample receptacle for receiving test sample. The second region comprises an electrophoretic medium that contains immobilized capture probes. The third region can be formed by excising the bottom support of the well creating an orifice, in some instances the microtiter well has a pointed tip which can be trimmed to provide an opening. This bottom orifice can optionally be covered using a porous membrane to provide support for the gel layer (second region). Preferably, this porous membrane has a molecular weight cutoff greater than 15,000 daltons. More preferably, the molecular weight cutoff is approximately 3,000 daltons. Preferably, the porous membrane should demonstrate negligible binding of nucleic acid molecules. This bottom orifice can be used to gain access to the third region which can be physically attached or detached from the well itself. The third region can comprise a collecting chamber which can contain buffer.

Specifically encompassed by the present invention is a method for purifying primer extension sequencing products from a primer extension sequencing reaction using a purification device described herein. The target molecule is one, or more, of the primer extension sequencing products formed during a particular stage of a DNA sequencing protocol. Typically, the primer extension sequencing product can have a size from about 20 to about 2000 nucleotides in length. For example, a DNA molecule that is destined for nucleotide sequencing can be placed into an appropriate sequencing vector, such as the M13 phage vector. Under suitable conditions well known to those skilled in the art, extension nucleic acid products can be produced using this vector, preferably using the cycle sequencing method. (See FIG. 1; Carothers, Biotechniques, 7:494-499 (1989), and Murray, Nucleic Acid Res., 17:8889 (1989)). Some of the reactants employed in this primer extension sequencing reaction are DNA Polymerase, primers, deoxynucleotides, and appropriate salts. Those skilled in the art will be familiar with standard DNA sequencing protocols. (See, Ausbel, F. M., et al. (eds), Current Protocols in Molecular Biology, vol.1, ch.7, (1995)). The target molecule (primer extension sequencing product) can subsequently undergo purification using a purification device.

In one embodiment of the present invention, a method for purifying multiple primer extension sequencing products which are formed by synthesizing target molecules (i.e., primer extension sequencing products) using both a first-end (e.g., near or at the 5′ end) and a second-end (e.g., near or at the 3′ end) of the DNA template simultaneously is described. Primer extension sequencing can occur in both directions of the template simultaneously. In this embodiment, a first-end primer and a second-end primer would be concurrently annealed to the template DNA allowing for extension in both direction using the one template DNA. The primer extension sequencing reaction would then occur and produce primer extension sequencing products arising from both the first-end and the second-end of the DNA template. The primer extension sequencing products can then be added to a purification device that can purify the target molecule based upon whether it was synthesized using the first-end primer or second-end primer.

In this embodiment, the purification device comprises at least two electrophoretic gel cartridges that can be brought together to form a continuity between the two cartridges. A gel cartridge is a device that can house and support an electrophoretic medium. The gel cartridges comprise electrophoretic medium containing capture probes immobilized within the medium. However, each cartridge comprises an electrophoretic medium containing different immobilized capture probes. For example, one cartridge can comprise an electrophoretic medium containing an immobilized capture probe that contains a nucleotide sequence which is substantially identical to a nucleotide sequence that lies adjacent, or close to, the first-end of the template (capture probe “A”), whereas, the second cartridge can comprise an electrophoretic medium which contains an immobilized capture probe that contains a nucleotide sequence which is substantially identical to a nucleotide sequence that lies adjacent, or close to, the second-end of the template (capture probe “B”). By “substantially identical to,” it is meant a nucleotide sequence with greater than 70% sequence identity and/or similarity (e.g., 75%, 80%, 85%, 90%, or 95% or greater homology). Initial search for substantially identical nucleotide sequences can be performed at NCBI against the GenBank (release 87.0), EMBL (release 39.0), and SwissProt (release 30.0) databases using the BLAST network service. Altshul, S. F., et al., Basic Local Alignment Search Tool, J. Mol. Biol., 215:403 (1990), the entire teachings of which are incorporated herein by reference in its entirety. Computer analysis of nucleotide sequences can be performed using MOTIFS and the FindPatterns subroutines of the Genetics Computing Group (GCG, version 8.0) software. Nucleotide comparisons can also be performed according to Higgins and Sharp (Higgins, D. G. and P. M. Sharp, Description of the Method used in CLUSTAL, Gene, 73:237-244 (1998)).

The two cartridges can be positioned in such a way as to allow for the migration of target molecules through one cartridge into the next cartridge.

For example, the cartridge that has the “A” capture probe is positioned such that it first receives the sample, and the second cartridge, which has the “B” capture probe, is positioned to receive the migrating sample from the first cartridge. If a test sample containing a heterogeneous mixture of target molecules (those that were synthesized using the first-end primer together with those that employed the second-end primer) is added to this purification device, then the target molecules can be purified or separated based upon the primer used to synthesize the target molecule. When an electric field is applied to the purification device, the target molecules in the test sample can undergo electrophoretic migration. Those target molecules that used the first-end primer for synthesis will be captured in the first cartridge containing “A” as capture probes (its appropriate capture probe), while those target molecules that used the second-end primer will migrate through the first cartridge and will subsequently be captured in the second cartridge containing “B” capture probes (its appropriate capture probe). Following electrophoretic migration, the cartridges can be separated and placed into separate collecting chambers, thereby allowing for the collecting of the first-end primer target molecules and the second-end primer target molecules separately.

Any electrophoretic matrix suitable for electrophoresis can be used for the methods of the present invention. Suitable matrices include acrylamide and agarose, both commonly used for nucleic acid electrophoresis. However, other materials may be used as well. Examples include chemically modified acrylamides, starch, dextrans, cellulose-based polymers. Additional examples include modified acrylamides and acrylate esters (for examples see Polysciences, Inc., Polymer & Monomer catalog, 1996-1997, Warrington, Pa.), starch (Smithies, Biochem. J., 71:585 (1959); product number S5651, Sigma Chemical Co., St. Louis, Mo.), dextrans (for examples see Polysciences, Inc., Polymer & Monomer Catalog, 1996-1997, Warrington, Pa.), and cellulose-based polymers (for examples see Quesada, Current Opin. in Biotechnology, 8:82-93 (1997)). Any of these polymers listed above can be chemically modified to allow specific attachment of capture probes for use in the present invention.

The capture probes of the instant invention are typically nucleic acids, modified nucleic acids, or nucleic acid analogs. The capture probes are complementary to the primer extension sequencing products, but not to the primer extension sequencing primer. Methods of coupling nucleic acids to create nucleic acid-containing gels are known to those of skill in the art. Nucleic acids, modified nucleic acids and nucleic acid analogs can be coupled to agarose, dextrans, cellulose, and starch polymers using cyanogen bromide or cyanuric chloride activation. Polymers containing carboxyl groups can be coupled to synthetic capture probes having primary amine groups using carbodiimide coupling. Polymers carrying primary amines can be coupled to amine-containing probes with glutaraldehyde or cyanuric chloride. Many polymers can be modified with thiol-reactive groups which can be coupled to thiol-containing synthetic probes. Many other suitable methods can be found in the literature. (For review see Wong, “Chemistry of Protein Conjugation and Cross-linking”, CRC Press, Boca Raton, Fla., 1993).

A variety of capture probes can be used in the methods of the present invention. Typically, the capture probes of the present invention comprise a nucleic acid (e.g., oligonucleotide) with a nucleotide sequence substantially complementary to a nucleotide sequence region contained within the target molecule wherein the target molecule hybridizes to the capture probe. It is important to note that the capture probe is not complementary to the primer used in the primer extension sequencing reaction. The complementarity of the capture probe to the target molecule need only be sufficient so as to specifically bind the target molecule and effectuate the purification of the target molecule in the reaction mixture. Probes suitable for use in the present invention comprise RNA, DNA, nucleic acid analogs (such as PNA), modified nucleic acids and chimeric probes of a mixed class comprising a nucleic acid with another organic component, e.g., peptide nucleic acids (PNA). Capture probes can be single-stranded or double-stranded nucleic acids. Typically, the length of a capture probe will be at least 5 nucleotides in length, more typically between 5 and 50 nucleotides, and can be as long as several thousand bases in length.

Methods for covalently attaching the capture probes described herein to polymerizable chemical groups have also been developed. When copolymerized with suitable mixtures of polymerizable monomer compounds, matrices containing high concentrations of immobilized nucleic acids can be produced. Examples of methods for covalently attaching nucleic acids to polymerizable chemical groups are found in U.S. Ser. No. 08/812,105, now U.S. Pat. No. 5,932,711; U.S. Ser. No. 08/971,845, and Rehman, F. N., et al., Nucleic Acid Res., 27:649-655 (1999), the teachings of which are herein incorporated by reference in their entirety.

For some methods, it may be useful to use composite matrices containing a mixture of two or more matrix forming materials, an example is the composite acrylamide-agarose gel. These gels typically contain from 2-5% acrylamide and 0.5%-1% agarose. In these gels the acrylamide provides the chief sieving function, but without the agarose, such low concentration acrylamide gels lack mechanical strength for convenient handling. The agarose provides mechanical support without significantly altering the sieving properties of the acrylamide. In such cases, it is preferred that the nucleic acid can be attached to the component that confers the sieving function of the gel, since that component makes the most intimate contacts with the solution phase nucleic acid target.

For many applications gel-forming matrices such as agarose and cross-linked polyacrylamide will be preferred. However, for capillary electrophoresis (CE) applications it is convenient and reproducible to use soluble polymers as electrophoretic matrices. Examples of soluble polymers that have proven to be useful for CE analysis are linear polymers of polyacrylamide, poly(N,N-dimethylacrylamide), poly(hydroxyethylcellulose), poly(ethyleneoxide) and poly(vinylalcohol) as described in Quesada (Current Opinion in Biotechnology, 8:82-93 (1997)). These soluble matrices can also be used to practice the methods of the present invention. It is particularly convenient to use the methods found in the application U.S. Ser. No. 08/812,105, now U.S. Pat. No. 5,932,711 entitled “Nucleic Acid-Containing Polymerizable Complex” for preparation of soluble polymer matrices containing immobilized capture probes. Another approach for attaching nucleic acid molecule probes to preformed polyacrylamide gels found in Timofeev, et al., Nucleic Acids Res., 24:3142-3148 (1996), can also be used to attach capture probes to prepolymerized soluble linear polyacrylamide.

Nucleic acids may be attached to particles which themselves can be incorporated into electrophoretic matrices. The particles can be macroscopic, microscopic, or colloidal in nature. (See Polyciences, Inc., 1995-1996 particle Catalog, Warrington, Pa.). Cantor, et al., U.S. Pat. No. 5,482,863 describes methods for casting electrophoresis gels containing suspensions or particles. The particles are linked to nucleic acids using methods similar to those described above mixed with gel forming compounds and cast as a suspension into the desired matrix form.

As defined herein, the term “nucleic acid” includes DNA (deoxyribonucleic acid) or RNA (ribonucleic acid). Nucleic acids referred to herein as “isolated” are nucleic acids separated away from the components of their source of origin (e.g., as it exists in cells, or a mixture of nucleic acids such as a library) and may have undergone further processing. Isolated nucleic acids include nucleic acids obtained by methods known to those of skill in the art. These isolated nucleic acids include substantially pure nucleic acids, nucleic acids produced by chemical synthesis, by combinations of biological and chemical methods and recombinant nucleic acids which are isolated.

“Nucleic acid analogs”, as used herein, include nucleic acids containing modified sugar groups, phosphate groups or modified bases. Examples of nucleic acids having modified bases, include, for example, acetylated, carboxylated or methylated bases (e.g., 4-acetylcytidine, 5-carboxymethylaminomethyluridine, 1-methylinosine, norvaline or allo-isoleucine). Such nucleic acid analogs are known to those of skill in the art. One example of a useful nucleic acid analog is peptide nucleic acid (PNA), in which standard DNA bases are attached to a modified peptide backbone comprised of repeating N-(2-aminoethyl)glycine units (Nielsen et al., Science, 254:1497-1500, (1991)). The peptide backbone is capable of holding the bases at the proper distance to base pair with standard DNA and RNA single strands. PNA-DNA hybrid duplexes are much stronger than equivalent DNA-DNA duplexes, probably due to the fact that there are no negatively charged phosphodiester linkages in the PNA strand. In addition, because of their unusual structure PNAs are very resistant to nuclease degradation. For these reasons, PNA nucleic acid analogs are useful for immobilized probe assays. It will be apparent to those skilled in the art that similar design strategies can be used to construct other nucleic acid analogs that will have useful properties for immobilized probe assays. Probes containing modified nucleic acid molecules may also be useful. For instance, nucleic acid molecules containing deazaguanine and uracil bases can be used in place of guanine and thymine-containing nucleic acid molecules to decrease the thermal stability of hybridized probes (Wetmur, Critical reviews in Biochemistry and Molecular Biology, 26:227-259 (1991)). Similarly, 5-methylcytosine can be substituted for cytosine if hybrids of increased thermal stability are desired (Wetmur, Critical reviews in Biochemistry and Molecular Biology, 26:227-259 (1991)). Modifications to the ribose sugar group, such as the addition of 2′-O-methyl groups can reduce the nuclease susceptibility of immobilized RNA probes (Wagner, Nature, 372:333-335 (1994)). Modifications that remove negative charge from the phosphodiester backbone can increase the thermal stability of hybrids (Moody et al. Nucleic Acids Res., 17:4769-4782 (1989); Iyer et al. J. Biol. Chem., 270:14712-14717 (1995)).

As defined herein, “substantially complementary” means that the nucleic acid molecule sequence of the capture probe need not reflect the exact nucleic acid molecule sequence of the microbial target molecule, but must be sufficiently similar in identity of sequence to hybridize with the target molecule under specified conditions. For example, non-complementary bases, or additional nucleic acid molecules can be interspersed in sequences provided that the sequences have sufficient complementary bases to hybridize therewith. Generally, the degree of complementarity using short capture probes (approximately 20 nucleotides in length) is approximately greater than 95%. For longer probes significantly less complementarity is required if there are contiguous segments of from about 15 to about 20 nucleotides in length being complementary to each other.

Specified conditions of hybridization can be determined empirically by those of skill in the art. For example, conditions of stringency should be chosen that significantly decrease non-specific hybridization reactions. Stringency conditions for nucleic acid hybridizations are explained in e.g., Current Protocols in Molecular Biology, Ausubel, F. M., et al., eds., Vol. 1, Suppl, 26, 1991; the teachings of which are herein incorporated by reference in their entirety. Factors such as probe length, base composition, percent mismatch between the hybridizing sequences, temperature and ionic strength influence the stability of nucleic acid hybrids. Stringent conditions, e.g., moderate, or high stringency, can be determined empirically, depending on part of the characteristics of the probe and microbial target molecule.

The features and other details of the invention will now be more particularly described and pointed out in the exemplification. It will be understood that the particular embodiments of the invention are shown by way of illustration and not as limitations of the invention. The principle features of this invention can be employed in various embodiments without departing from the scope of the invention.

Exemplification

### Gel-Based DNA Isolation and Elution

Sequencing products were prepared using the Thermo Sequenase™ DYEnamic direct cycle sequencing kit with −21 M13 forward primer (5′-dyel-spacer-TGT*AAAACGACGGCCAGT-3′ [SEQ ID No. 1]), where * indicates the position of base modification with one of four fluorescence energy transfer dyes according to the manufacturer's instructions (Amersham Pharmacia Biotech, Piscataway, N.J.). Each of four reactions was prepared by mixing 1 mL of M13mp18 single-stranded DNA (0.25 mg/mL, New England BioLabs catalog #404-C), 14 mL of distilled H₂O, and 2 mL of the manufacturer's reaction mixture. Each of the four reactions used a different dye-labeled primer and a different ddNTP nucleotide mixture. These four tubes were then placed in a thermal cycler (PTC100, MJ Research, Watertown, Mass.) and subjected to 30 cycles of 95° C. for 30 seconds, 45° C. for 15 seconds, and 70° C. for 30 seconds. The four reactions were then pooled (100 mL total volume) and 11 mL of loading buffer was added (2.5% wt/vol Xylene Cyanol, 2.5% wt/vol Bromophenol Blue, 20 mM EDTA, pH 8.0, 15% (wt/vol) Ficoll 400,000 average molecular weight in a total of 10 mL distilled H₂O).

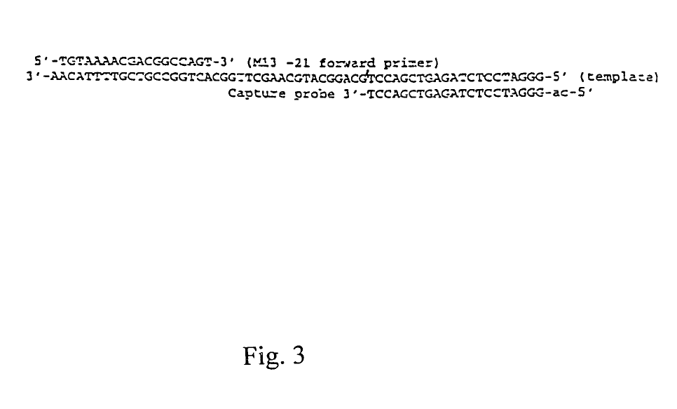

Polyacrylamide gels for electrophoretic hybridization purification were cast in standard micropipette tips for 1-200 μL micropipettes (Fisher Brand yellow tips for Gilson P200, Fisher Scientific, Pittsburgh, Pa.). For the purification step, two gel tips were stacked so that the sequencing reaction could be subjected to electrophoresis through each tip sequentially in one step. (See FIG. 3a). The gel in the upper tip comprised a 20 μL 5% polyacrylamide gel (29:1 monomer:bis wt/wt) cast in 1× TBE buffer (89 mM Tris-Borate pH 8.3, 2 mM EDTA (Bio-Rad). This upper gel is designed to trap the high molecular weight M13 template DNA which has negligible electrophoretic mobility under the conditions used for capture of the extension sequencing products. Removal of the high molecular weight template improves quality of sequencing results on capillary electrophoresis instruments such as the Megabase from Molecular Dynamics (Sunnyvale, Calif.).

The gel in the lower tip is the same as that of the upper tip, except that it contains an immobilized nucleic acid molecule capture probe (5′-acrylamide-GGG ATC CTC TAG AGT CGA CCT 3′ [SEQ ID No. 2]) at a concentration of 10 μM (referring to nucleic acid molecule strands). The capture probe is complementary to a sequence within the extension products that is located immediately 3′ of the sequencing primer, as shown in FIG. 3.

As shown if FIG. 3, the cloned insert to be sequenced is located on the 5′ side of the template region shown. Thus, as shown in the diagram, the capture probe is complementary to the extended sequencing products, but not to the sequencing primer. In this way, electrophoresis of the extension sequencing products through the gel of the lower tip will allow hybridization capture of the extension products without impeding electrophoresis of the excess primers through the tip.

The capture probe was modified with a 5′-acrylamide group using an acrylamide phosphoramidite (Acrydite™, Mosaic Technologies, Boston, Mass.). The probe was immobilized on the gel matrix by adding it to the unpolymerized acrylamide mixture and allowing it to copolymerize directly in the gel tip.

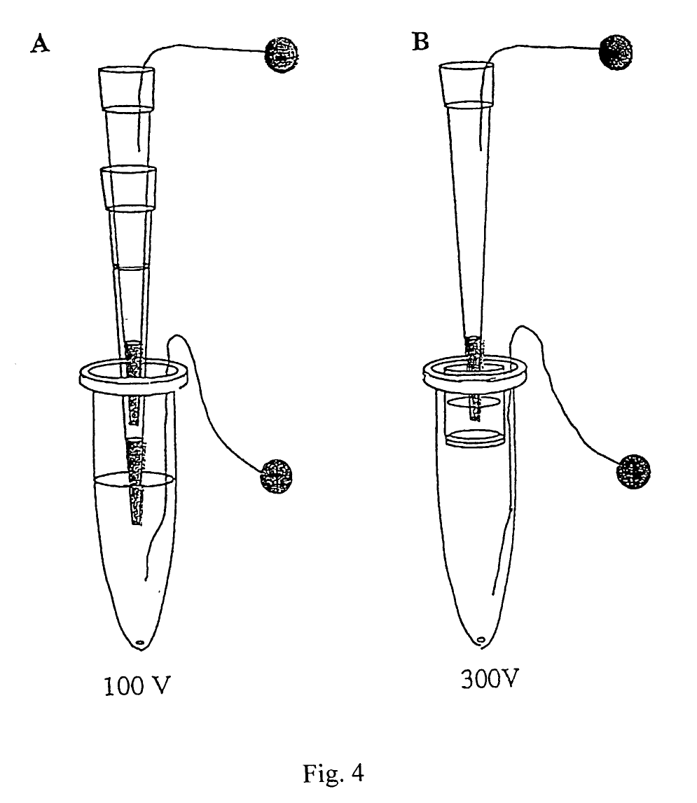

The tips were stacked as shown in FIG. 4a, where the probe-containing tip is on the bottom. The space between the two tips is filled with electrophoresis buffer (1× TBE which is 89 mM Tris-borate, pH 8.3, 2 mM EDTA), as well as the space above the upper gel tip. The lower tip is immersed in a buffer-filled 1.5 mL microcentrifuge tube. Separate platinum electrodes are placed in the buffer above the gel in the higher tube and in the buffer in the microcentrifuge tube. The upper electrode is connected to a negative lead of the power supply, while the lower electrode is attached to a positive lead.

The upper tip of the device shown in FIG. 4a was loaded with 75 μL of the pooled sequencing reaction in 15 μL aliquots every 10 minutes for one hour, while subjecting the tips to electrophoresis at an applied field of 100 V throughout the loading process. The field was applied for an additional 3 hours to ensure that all of the sequencing products become trapped on the gel in the lower tip. The primers, which are not complementary to the immobilized capture nucleic acid molecule probes in the lower gel, nucleotides, and excess salts pass through the gel into the lower tube.

Following electrophoresis, the upper gel tip containing the slow-moving template was discarded and the lower gel tip was then placed into a second apparatus, depicted in FIG. 4b. The lower end of the tip is placed into an electrophoresis buffer held in an ultrafiltration device with a 3000 Dalton molecular weight cutoff membrane on its bottom surface (Microcon 3, Amicon/Millipore, Bedford, Mass.). The ultrafiltration unit was partially immersed in a 1× TBE-filled microcentrifuge tube containing a positively charge platinum electrode. A negatively-charged electrode was immersed the buffer (1× TBE) above the gel in the tip. The ultrafiltration membrane was used to prevent the migration of the eluted sequencing products onto the electrode, where they would be damaged by electrochemical reactions. To elute the sequencing products, a field of 300 V was applied to the device for 3 minutes. This voltage was sufficient to elute the sequencing products from the gel capture probes and cause it to collect in the ultrafiltration unit.

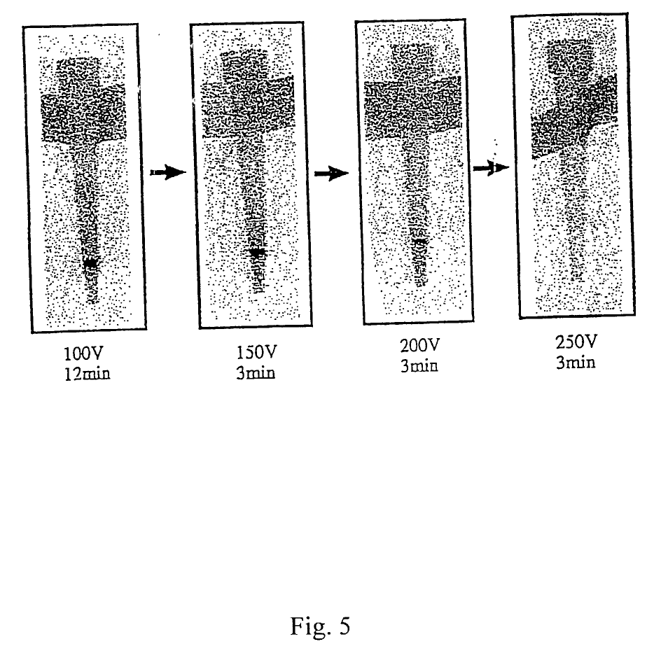

FIG. 5 shows the effects of varying the elution voltage. Sequencing products were captured and purified by electrophoretic hybridization capture as described above. The tip was then subjected to the indicated electrophoresis conditions, and then scanned in a fluorescence imaging device (Fluoroimager 595, Molecular Dynamics, Sunnyvale, Calif.) to visualize the fluorescent sequencing products. As seen, voltages above 250 V cause complete elution of the fluorescent sequencing products.

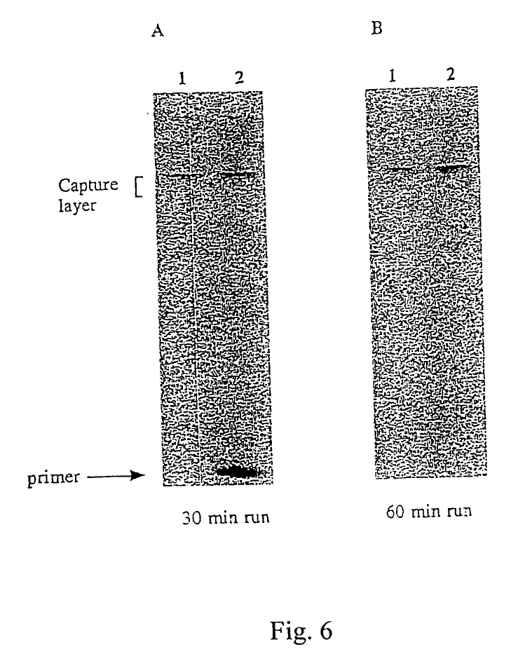

To characterize the eluted products, samples of purified and crude sequencing products were subjected to electrophoresis in a polyacrylamide gel containing a discrete layer of gel immobilized capture probe arranged as a horizontal band across the width of the gel (see “Capture layer” in FIG. 6). The gel was composed of 5% polyacrylamide (29:1 monomer:bis wt/wt), 1× TBE. The capture layer contained the same polyacrylamide and buffer with 10 μM of the 5′-acrylamide capture probe (5′-acrylamide-GGG ATC CTC TAG AGT CGA CCT 3′ [SEQ ID No. 6]). The samples were subjected to electrophoresis run at 150 Volts for 30 minutes (FIG. 6a) and 60 minutes (FIG. 6b). Lane 1 contains 15 μL of the sample that had been purified by electrophoretic capture and elution, and lane 2 contains 5 μL of the unpurified sequence product. FIG. 6a shows that the hybridization-purified product (lane 1) has been purified away from the excess primers, which are seen in the unpurified sample at the bottom of lane 2.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method for purifying target molecules from a primer extension sequencing reaction using a purification device comprising the following steps:
(a) introducing the primer extension sequencing reaction mixture into a purification device comprising an electrophoretic medium containing immobilized capture probes, wherein the immobilized capture probes are selected from the group consisting of nucleic acids, modified nucleic acids and nucleic acid analogs;
(b) subjecting the electrophoretic medium of step (a) to an electric field resulting in the electrophoretic migration of one, or more, target molecules into at least one region of the electrophoretic medium containing immobilized capture probes, wherein the target molecules bind to the immobilized capture probes and non-target molecules continue to migrate under the influence of the electric field, thereby separating the target molecules from the non-target molecules of the primer extension sequencing reaction mixture;
(c) imposing conditions on the electrophoretic medium that dissociate the targets and their complementary capture probes;
(d) applying an electric field while maintaining the dissociating conditions within the electrophoretic medium, thereby causing the dissociated target molecules to exit the electrophoretic medium by electrophoretic migration; and
(e) collecting the target molecules which have exited the electrophoretic medium.

2. The method of claim 1, wherein the purification device is a microtiter plate.

3. The method of claim 2, wherein the microtiter plate comprises multiple wells.

4. The method of claim 3, wherein the number of wells contained within the microtiter plate is selected from the group consisting of: 6, 12, 48, 96 and 384.

5. The method of claim 1 wherein in step (c), a sufficient voltage is applied to release the target molecule from its complementary capture probe, and wherein the target molecule continues electrophoretic migration under the influence of an electric field and exits the electrophoretic medium, and wherein it collects in a collecting chamber.

6. The method of claim 5, wherein the polarity of the electric field is reversed, wherein the released target molecule will migrate back toward the test sample receptacle and wherein it is subject to collection.

7. The method of claim 1, wherein the capture probe is a nucleic acid molecule.

8. The method of claim 7, wherein the capture probe is complementary to the primer extension sequencing product.

9. The method of claim 8, wherein the capture probe is from about 20 to about 2000 nucleotides in length.

10. A method for purifying multiple sets of primer extension sequencing reaction products which are formed by synthesizing multiple sets of primer extension sequencing reaction products comprising the following steps:
(a) introducing the multiple sets of primer extension sequencing reaction products into a purification device comprising at least two cartridges, wherein each cartridge comprises an electrophoretic medium containing at least one unique set of immobilized capture probes, and wherein the immobilized capture probes are selected from the group consisting of nucleic acids, modified nucleic acids and nucleic acid analogs;
(b) subjecting the electrophoretic media of step (a) to an electric field resulting in the electrophoretic migration of one, or more, sets of primer extension sequencing reaction products into the cartridges of step (a), wherein target molecules in each set of primer extension sequencing reaction products bind to a substantially complimentary set of immobilized capture probes and non-target molecules continue to migrate under the influence of the electric field, thereby separating the target molecules from the non-target molecules in each set of the primer extension sequencing reaction products;
(c) imposing conditions on the electrophoretic medium that dissociate the target molecules and their complementary capture probes;
(d) applying an electric field while maintaining the dissociating conditions within the electrophoretic medium, thereby causing the dissociated target molecules to exit the electrophoretic medium by electrophoretic migration; and
(e) collecting each set of primer extension sequencing reaction target molecules which exit the electrophoretic medium.

11. The method of claim 10, wherein the purification device is a microtiter plate.

12. The method of claim 11, wherein the microtiter plate comprises multiple wells.

13. The method of claim 12, wherein the number wells contained within the microtiter plate is selected from the group consisting of: 6, 12, 48, 96 and 384.

14. The method of claim 10 wherein in step (c), a sufficient voltage is applied to release the target molecule from its complementary capture probe, and wherein the target molecule continues electrophoretic migration under the influence of an electric field and exits the electrophoretic medium, and wherein it collects in a collecting chamber.

15. The method of claim 14, wherein the polarity of the electric field is reversed, wherein the released target molecule will migrate back toward the test sample receptacle and wherein it is subject to collection.

16. The method of claim 10, wherein the capture probe is a nucleic acid molecule.

17. The method of claim 17, wherein the capture probe is complementary to the primer extension sequencing product.

18. The method of claim 17, wherein the capture probe is from about 20 to about 2000 nucleotides in length.

19. A method for purifying target molecules from a primer extension sequencing reaction using a purification device comprising the following steps:
(a) introducing the primer extension sequencing reaction mixture into a purification device comprising an electrophoretic medium containing capture probes modified with a 5′-acrylamide moiety and selected from the group consisting of nucleic acids, modified nucleic acids and nucleic acid analogs, wherein the capture probes are immobilized in the electrophoretic medium by a covalent bond between the 5′-acrylamide moiety and the electrophoretic medium;
(b) subjecting the electrophoretic medium of step (a) to an electric field resulting in the electrophoretic migration of one, or more, target molecules into at least one region of the electrophoretic medium containing immobilized capture probes, wherein the target molecules bind to the immobilized capture probes and non-target molecules continue to migrate under the influence of the electric field, thereby separating the target molecules from the non-target molecules of the primer extension sequencing reaction mixture;
(c) imposing conditions on the electrophoretic medium that dissociate the targets and their complementary capture probes;
(d) applying an electric field while maintaining the dissociating conditions within the electrophoretic medium, thereby causing the dissociated target molecules to exit the electrophoretic medium by electrophoretic migration; and
(e) collecting the target molecules which have exited the electrophoretic medium.

20. The method of claim 19, wherein the capture probes are immobilized in the electrophoretic medium by copolymerizing the 5′-acrylamide moiety with the electrophoretic medium.
